(19) United States
(12) Patent Application Publication
(10) Pub. No.: US 2004/0024042 A1
(43) Pub. Date: Feb. 5, 2004

(54) COX2 inhibition in the prevention and treatment of autosomal dominant polycystic kidney disease

(75) Inventor: Matthew Breyer, Nashville, TN (US)

Correspondence Address: FULBRIGHT & JAWORSKI L.L.P., 600 CONGRESS AVENUE, SUITE 2400, AUSTIN, TX, 78701-3271, US

(73) Assignee: Vanderbilt University

(21) Appl. No.: 10/405,959

(22) Filed: Apr. 2, 2003

Related U.S. Application Data
(60) Provisional application No. 60/370,138, filed on Apr. 2, 2002.

(51) Int. Cl. A61K 31/415; A61K 31/365

(52) U.S. Cl. 514/406; 514/473

(57) ABSTRACT

The present invention provides a new therapeutic approach for autosomal dominant polycystic kidney disease (ADPKD). Cyclooxygenase 2 (COX2) inhibitors are used, alone or in combination with other drugs, to prevent or limit early stage cyst formation.

### CROSS-REFERENCE(S) TO RELATED APPLICATION(S)

This application is related to, and claims a benefit of priority under 35 U.S.C. 119(e) and/or 35 U.S.C. 120 from, copending provisional U.S. Ser. No. 60/370,138, filed Apr. 2, 2002, the entire contents of which are hereby expressly incorporated by reference for all purposes.

### BACKGROUND OF THE INVENTION

1. Field of the Invention

The present invention relates generally to the fields of nephrology and pharmaceutical biology. More particularly, it concerns the use of cyclooxygenase 2 (COX2) inhibitors to prevent and treat autosomal dominant polycystic kidney disease (ADPKD).

2. Description of Related Art

Autosomal dominant polycystic kidney disease is one of the most common genetic diseases occurring in 1:500 to 1:1000 live births (Gabow, 1993). ADPKD is the primary diagnosis in ˜10% of patients with ESRD in the US and there are approximately 400,000 patients with varying degrees of CR1 due to ADPKD in the US not yet on dialysis (System, 1999). With better overall health care and longer life spans, it is likely that over the next decade many more patients with ADPKD will survive longer and require renal replacement therapy. Despite advances in retarding the progression of renal disease in diabetic nephropathy (Lewis et al., 1993; Lewis et al., 2001), proteinuric nephropathies (Maschio et al., 1996; Ruggenenti et al., 2000; Ruggenenti et al., 2000) and hypertensive nephrosclerosis (Agodoa et al., 2001), there have been no interventions clearly identified to slow or halt the relentless progression of ADPKD to end stage renal disease.

As compared to other common causes of renal failure, the pathogenic events conspiring to cause renal failure in ADPKD are unique. In contrast to diabetic nephropathy or focal segmental glomerulosclerosis (FSGS), where significant proteinuria invariably accompanies progression of the disease, in ADPKD glomerular architecture remains intact until late in its course. Therefore the use of proteinuria as a surrogate for severity of ADPKD is at best a weak marker, and at worst, misleading (Chapman et al., 1994; Contreras et al., 1995). In fact, the occurrence of nephrotic range proteinuria in ADPKD is so rare as to be an indicator of the presence of a second superimposed renal disease (Contreras et al., 1995). These differences have focused therapeutic efforts targeting the molecular basis for cyst formation or reducing the rate of cyst expansion (Davis et al., 2001; Grantham, 1997; Qian et al., 2001). Renal failure develops in ADPKD as a consequence of disordered renal architecture resulting from progressive expansion of epithelial cysts and accompanying reabsorption and remodeling of the normal renal tissue interposed between expanding cysts.

Renal cystogenesis may be conceptualized as a two-step process: initiation of cyst formation followed by progression of cyst expansion. The molecular events initiating cyst formation are incompletely understood. Models of cystogenesis must account for late development and focal cyst occurrence. Recent work suggests cyst formation is initiated as a result of a random somatic mutation of the remaining normal PKD allele in patients with germline disruption of one PKD allele (i.e., PKD1+/−) (Koptides et al., 1999; Qian et al., 1996). Analysis of the PKD alleles in cystic cells from ADPKD patients has revealed a loss of heterozygosity (LOH) or intragenic mutations involving the non-affected PKD1 allele in approximately 20% of renal cysts (Qian et al., 1996). Conversely, the detection of polycystin (PKD1) and PKD2 proteins by immunohistochemistry within most cysts is at odds with the hypothesis of the complete loss PKD1 expression within each cyst (Ong et al., 1999). Missense mutations, altered protein-protein interactions, or unexplored events including epigenetic alterations (Jackson-Grusby et al., 2001) in gene expression may play explain this discrepancy between the LOH model and presence of the PKD1 protein, polycystin.

While mutations in PKD1 or PKD2 initiate renal cyst formation, multiple factors modify the rate of cyst enlargement and progression of renal failure. Cyst growth involves at least three pathogenic mechanisms: enhanced cell proliferation, cell dedifferentiation with fluid secretion into the cyst lumen, and interstitial fibrosis (Grantham, 1997). Other emerging concepts include the importance of epithelial-stromal interactions (Arias, 2001; Liotta and Kohn, 2001; Qian et al., 2001) and induction of angiogenesis (Bello-Reuss et al., 2001). Therapeutic interventions may therefore target not only the abnormal epithelium, but also the surrounding stromal tissue that supports its growth.

Accelerated epithelial cell proliferation has been uniformly seen in both human ADPKD cysts and animal models of renal cystic disease (Nadasdy et al., 1995; Ramasubbu et al., 1998), suggesting that epithelial cell proliferation is an essential component of cystogenesis. Hyperplastic polyps are commonly observed within the cyst wall (Bernstein et al., 1987), and cyst formation is accompanied by enhanced apoptosis in ADPKD epithelium (Woo, 1995). Parallels can be drawn between the benign epithelial proliferation in PKD and cell growth observed in intestinal polyposis (Kinzler and Vogelstein, 1996; Powell et al., 1993). As in ADPKD, familial adenomatous polyposis (FAP) represents an autosomal dominant disease associated with benign (at least initially) accelerated epithelial proliferation of intestinal cells. Vogelstein and Kinzler's group identified the relevant mutation in a novel gene designated APC (adenomatous polyposis coli) (Su et al., 1993). As in ADPKD, FAP is an autosomal dominant but the of polyps develop sporadically. This has been demonstrated to be associated with loss of heterozygosity (LOH) due to somatic cell mutations resulting in loss of the remaining normal somatic cell APC allele, much as has been proposed for ADPKD. Importantly the signaling pathway activated by the APC protein and polycystin-1 may be similar since its been shown that both proteins interact with the β-catenin-nuclear signaling pathway, altering gene expression and activating cell proliferation (Kim et al., 1999; Korinek et al., 1997; Morin et al., 1997; van Adelsberg, 2000). These considerations provide a conceptual basis for utilizing similar approaches for treating intestinal polyposis and ADPKD.

Recent clinical trials demonstrated an unexpected role for COX2 activity in promoting growth of colonic polyps (Steinbach et al., 2000) and that NSAIDs prevent colon cancer (Giovannucci et al., 1995; Marcus, 1995; Shiff and Rigas, 1999). In a randomized, double-blind, placebo-controlled study, patients with FAP treated with nonselective NSAIDs or COX2 inhibitors exhibit a striking decrease in number and size of polyps (Giardiello et al., 1993; Steinbach et al., 2000). Similarly COX2 knockout mice or mice treated with either COX2 inhibitor, celecoxib or rofecoxib, exhibit a marked reduction in polyp formation was seen mouse models of FAP (Oshima et al., 1996, Jacoby, 2000 [#5288]; Oshima et al., 2001). In both human and mouse polyps COX2 expression is increased primarily in interstitial cells, possibly infiltrating macrophages, rather than in the polyp epithelium (Bamba et al., 1999; Chapple et al., 2000; Hardwick et al., 2001). These observations are consistent with a model whereby stromal COX2 derived prostanoids exerts a paracrine effect promoting epithelial growth and/or angiogenesis (Masferrer et al., 2000; Williams et al., 1999). Importantly as described below, in preliminary data the inventor sees a similar increase in interstitial COX2 expression in human polycystic kidney disease, further underscoring the similarities in between ADPKD and FAP.

In addition to their capacity to interfere with inflammation and angiogenesis, COX2 inhibitors may have deleterious effects on renal function (Breyer and Harris, 2001; Harris and Breyer, 2001). Like other nonselective NSAIDs, COX2 inhibitors may enhance renal salt retention with resultant edema and hypertension (Brater, 1999; Breyer and Harris, 2001). Of note, these deleterious effects of NSAIDs on blood pressure are seen primarily with low renin hypertension (Jackson, 1989). In contrast, COX2 inhibition actually reduces blood pressure when hypertension is due to increased renin release (Harding et al., 2000; Imanishi et al., 1989; Wang et al., 1999). This may be directly relevant to their use in ADPKD, where hypertension is associated with inappropriate activation of the renin angiotensin system (Chapman et al., 1990; Ecder and Schrier, 2001).

Control of hypertension has been a mainstay of therapeutic intervention in kidney disease, forestalling progression to renal failure from all causes including ADPKD (Ecder and Schrier, 2001; Gabow et al., 1992; Geberth et al., 1995). Indeed this has been a major impetus for treating ADPKD patients with ACEI as well as other anti-hypertensives (Ecder et al., 2001; Ecder and Schrier, 2001). However in contrast to diabetic nephropathy and other proteinuric nephropathies, ACEIs afford no specific theoretical advantage in ADPKD over other anti-hypertensives, vis-á-vis glomerular hemodynamics. Animal models provide some support for implementing RAS inhibition as a therapeutic strategy in ADPKD (Keith et al., 1994; Kennefick et al., 1999; Ogborn et al., 1995). Use of ACEI or ARBs in rats with autosomal dominant polycystic kidney (Han: SPRD (Cy/+)), suggest a beneficial effect on progression as compared to placebo, but no clear advantage above and beyond that seen with conventional anti-hypertensives (Keith et al., 1994; Kennefick et al., 1999; Ogbom et al., 1995). Nonetheless, cyst enlargement in ADPKD has been postulated to cause local renal ischemia, and some studies suggest that renal renin production is, indeed, increased in ADPKD (Graham and Lindop, 1988; Torres et al., 1992). This provides a theoretical advantage for the use of drugs which suppress the renin, angiotensinaldosterone system (RAAS) in ADPKD (Ecder and Schrier, 2001; Fick and Gabow, 1994).

Despite the preceding considerations, few available studies in humans support a particular advantage of ACEIs in ADPKD but all suffer from inadequate size. A large cohort study looking at the effect of ACEI on the rate of doubling of serum creatinine in non-diabetic chronic renal insufficiency, found a highly significant protective effect in all subjects diseases except those with ADPKD (Maschio et al., 1996). Similarly although dramatic benefit of ACEI was demonstrated in the Gisen study, looking at non-diabetic proteinuric renal diseases, patients with ADPKD were not a subgroup which benefited by ACEI (Ruggenenti, 2000 [#4742]). Conversely, a recent retrospective analysis of 33 ADPKD patients treated with anti-hypertensives suggested that those patients treated with diuretics had a more rapid decline in renal function than those patients treated with ACEI (Ecder et al., 2001). All the available studies in humans with ADPKD examining the efficacy of ACEI have small sample sizes and are underpowered to protect against either type 1 or type 2 errors.

Other therapeutic interventions have similarly been shown to be ineffective in the ADPKD patients enrolled. The Modification of Diet in Renal Disease (MDRD) study represents the largest randomized prospective controlled clinical trial following progression of renal disease in patients with ADPKD. Of the 840 participants, 200 had ADPKD (Klahr et al., 1995; Klahr et al., 1994). Using careful iothalamate measures of glomerular filtration rate (GFR) the MDRD was unable to demonstrate a beneficial effect of assignment to a low protein diet group or to a low blood pressure (MAP <92 mnhg) group on the rate of decline of GFR in ADPKD participants. The lack of efficacy of these interventions must be interpreted with caution since patients with ADPKD only represented a subgroup of a quarter of the patients enrolled in this trial. The efficacy of ACEI in patients with ADPKD has not been similarly prospectively evaluated in a study with adequate sample size.

### SUMMARY OF THE INVENTION

Thus, in accordance with the present invention, there is provided a method of preventing or treating autosomal dominant polycystic kidney disease (ADPKD) in a subject comprising administering to the subject an effective amount of a cyclooxygenase 2 (COX2) inhibitor. The COX2 inhibitor is selected from the group consisting of celecoxib, rofecoxib, paracoxib, valdecoxib, etoricoxib, meloxicam, or nimesulide. The method may comprise preventing ADPKD in a subject prior to the development of symptomatic renal disease, such as administration of said COX2 inhibitor beginning at early adulthood. The subject at risk of ADPKD may be determined by family history, renal imaging study and/or genetic screening. Alternatively, the method may comprise treating ADPKD in a subject exhibiting symptomatic renal disease and comprises slowing or halting disease progression. The subject may also suffers from chronic renal insufficiency. The dose of the COX2 inhibitor may be adjusted to avoid unwanted kidney blood flow effects, thereby maintaining normal renal function and blood pressure.

The method may further comprise administering to the subject a second drug, such as anti-hypertensive, for example, an ACE inhibitor, an angiotensin receptor blocker, or an aldosterone receptor antagonists. The subject may be a non-human animal or a human. The method may also further comprising monitoring one or more conditions in the subject following administration of said COX2 inhibitor, such as blood pressure, serum creatinine, blood urea nitrogen, urinary protein excretion, or glomerular filtration rate. The subject may also receive or have received therapeutic nephrectomy.

### DESCRIPTION OF ILLUSTRATIVE EMBODIMENTS

Autosomal dominant polycystic kidney disease (ADPKD) is one of the most common genetic disorders in man, affecting more than 1/1000 people world-wide. ADPKD is the primary diagnosis in 5-10% of the endstage renal disease population in the U.S. Renal cysts develop progressively and enlarge slowly during life. Patients with this disease generally live normal productive lives until the fourth of fifth decade when the cystic destruction of the kidney becomes so extensive that renal failure ensues. At this time dialysis or transplantation is required. Despite the ability to diagnose ADPKD early in life, there is little therapeutically to offer patients to forestall their predestined progression to renal failure in the fifth to sixth decades. Development of strategies to retard cyst expansion and proliferation represents an important approach to preventing renal failure.

COX2 is an immediate early growth response gene associated with cell division. Its aberrant expression is associated with epithelial cell proliferation and dedifferentiation in a variety of epithelial tumors including non-malignant colonic polyposis. In ADPKD, epithelial cell proliferation is an invariant component of cystogenesis, and micropolyps can be observed in the wall of cysts. Some cytotoxic agents, such as Taxol, slow progression of PKD in animal models, however because of its severe toxicity, its utility in human PKD is unlikely. Other more benign and prolonged therapies are being sought.

Preliminary data from the inventor's laboratory shows renal COX2 expression is increased in kidneys from both in human animal models of polycystic kidney. They have examined the expression of COX2 in kidneys harvested from patients following therapeutic nephrectomy for ADPKD as well as tissue removed for acquired PKD, revealing increased expression of COX2 mRNA (nuclease protection) and protein (by immunoblot) in the two patients versus for control kidneys. Immunohistochemistry expression localized increased COX2 expression to interstitial cells. Thus, the inventor proposes that COX2 inhibitors will prevent or limit cyst growth in patients with ADPKD, much in the same way as they are used in the rarer disease Familial adenomatous polyposis.

I. COX2 Inhibitors

Various COX2-specific and COX2-selective inhibitors are currently being developed. The of the most commonly prescribed of this class of drug are celecoxib and rofecoxib. Each of these drugs are discuss below. Other COX-2 selective inhibitors include paracoxib, valdecoxib, etoricoxib, meloxicam and nimesulide.

A. Celecoxib

Celecoxib, sold by Searle under the trade name CELBREX™, is chemically designated as 4-[5-(4-methyphenyl)-3-(trifluoromethyl)-1H-pyrazol-1-yl] benzenesulfonamide. The empirical formula is C₁₇H₁₄F₃N₃O₂S, and the molecular weight is 381.38. CELEBREX® is marketed in 100 or 200 mg oral capsules. Celecoxib exhibits anti-inflammatory, analgesic and antipyretic activities in various animal models.

1. Pharmacokinetics

Peak plasma levels of celecoxib are roughly 3 hours after an oral dose. When take with a high fat meal, plasma levels were delayed about 1-2 hours, with an increase in total absorption of 10-20%. Aluminum or magnesium containing antacids resulting in a decrease in plasma concentrations. Celecoxib is highly protein bound with the clinical dose range, with in vitro studies indicating that albumin and alpha₁-acid glycoprotein being the major bound species. Cytochrome P450 2C9 is the major metabolizing enzyme of celecoxib. The three primary metabolites are the alcohol, the corresponding carboxylic acid and its glucuronide conjugate; these metabolites are inactive as COX-1 and COX-2 inhibitors. Following a single dose, 57% of the dose was excreted in feces, and 27% in the urine. The effective half-life is roughly 11 hours under fasted conditions.

2. Patient Populations

Geriatric patients had high maximal serum concentrations, and elderly male had high concentrations than elderly females. For elderly patients of less than 50 kg, lower doses should be used initially. Blacks show higher serum concentrations than Caucasians. Hepatic insufficiency increases serum concentration, while renal insufficiency decreases concentration.

3. Drug Interactions

Patients should be questioned regarding the use of drugs that inhibit cytochrome P450 2C9. Specific potential drug interactions include fluconazole and lithium, and possibly furosemide and ACE inhibitors.

4. Side Effects and Contraindications

Side effects for NSAIDs typically include gastroduodenal and gastrointestinal irritation. However, celecoxib shows far less of these effects than other NSAIDs. Other possible side effects include anaphylactoid reactions, although none have been reported for celecoxib. It also should be avoided for patients with advanced renal disease and pregnant mothers.

B. Rofecoxib

Rofecoxib (VIOXX®) is a furanone derivative (molecular wt=314.36). It a nonsteroidal anti-inflammatory drug (NSAID) with a highly selective cyclooxygenase-2 (COX-2) inhibitory action. It possesses anti-inflammatory, analgesic, and antipyretic activities. Rofecoxib is indicated for relief of the signs and symptoms of osteoarthritis, management of acute pain in adults and treatment of primary dysmenorrhea. It is available as oral tablets (12.5 mg and 25 mg) and an oral suspension (12.5 mg or 25 mg per 5 mL).

1. Dose and Administration

For osteoarthritis, the recommended starting dose is 12.5 mg qday. The maximum recommended daily dose is 25 mg. The drug may be taken with or without food.

2. Pharmacokinetics

Oral bioavailability >90%; peak plasma concentration (Cₘₐₓ) after a 25-mg dose=0.2 μg/mL; Cₘₐₓ occurs 2-3 hrs after dose ingestion. Steady state level is reached after 4 days of administration of 25 mg qday; Cₛₛₘₐₓ=0.32 μg/mL. The drug tends to accumulate with repeated administration (accumulation factor=1.67). Food had no significant effect on either Cₘₐₓ or extent of absorption (AUC) of rofecoxib. Antacids reduce extent of absorption by 10-15% and Cₘₐₓ by 20%. Compared to young subjects, elderly patients (>65 yrs) showed a 34% increase in bioavailability. Race, gender, and mild liver disease have little or no effect on AUC. Patients with moderate hepatic insufficiency (Child-Pugh score 7-9) may have a significantly higher AUC (↑69%) and may require dose reduction. Plasma protein binding=87%; Cd=90 L. Animal studies showed that rofecoxib is able to cross the placenta and the blood-brain barrier.

Rofecoxib is metabolized mainly through reduction by cytosolic enzymes to inactive dihydro derivatives. Metabolic elimination of rofecoxib appears to be a nonlinear process due to saturable metabolic capacity. This capacity is enhanced by non-specific inducers like rifampin. Rofecoxib metabolites are excreted mainly in the urine, with <1% of the dose excreted unchanged in the urine. Rofecoxib is not a substrate for the P450 system, but it is a “mild inducer” of the 3A4 isoform. The use of rofecoxib in advanced renal disease is not recommended at present because of the lack of safety information. Rofecoxib is excreted in the milk of lactating animals and was found to be harmful to nursing infants. It is not known whether the same occurs in humans. However, it is use in lactating mothers is not recommended at the present.

3. Side Effects

Rofecoxib, like other NSAIDs, should be prescribed with extreme caution in patients with a prior history of ulcer disease or gastrointestinal bleeding. Most spontaneous reports of fatal GI events are in elderly or debilitated patients. Rofecoxib should not be given to patients with the aspirin triad. This symptom complex typically occurs in asthmatic patients who experience rhinitis with or without nasal polyps, or who exhibit severe, potentially fatal bronchospasm after taking aspirin or other NSAIDs.

4. Drug Interactions

Concomitant administration of low-dose aspirin with rofecoxib may result in an increased rate of GI ulceration or other complications, compared to use of rofecoxib alone. Although rofecoxib is not a substrate for the P450 system, non-specific enzyme inducers (e.g., rifampin) or inhibitors (e.g., cimetidine) are likely to affect its metabolic clearance. Rofecoxib is a mild inducer of Cytochrome P450 3A4. Therefore, it may reduce the blood level of such drugs as cyclosporine or tacrolimus that are metabolized by this system. A 30% reduction of the AUC of midazolam was observed with rofecoxib (25 mg daily). Rofecoxib may inhibit warfarin metabolism and enhance its anticoagulant effect (↑INR by about 10%).

II. Combination Therapies

In order to increase the effectiveness of a COX2 inhibitor therapy for ADPKD, it may be desirable to combine such inhibitors with other agents effective in the treatment of ADPKD, such as anti-hypertensive agents. This process may involve administering the agents at the same time. This may be achieved by administration of a pharmacological formulation that includes both agents, or by administration of two distinct compositions or formulations, at the same time.

Alternatively, the “other” drug may precede or follow the COX2 inhibitor treatment by intervals ranging from minutes to weeks. One may administer both modalities within about 6-12 hr of each other, 12-24 hr of each other, or 24-48 hr of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

Various combinations may be employed, COX2 inhibitor therapy is “A” and the other agent is “B”:

A/B/A B/A/B B/B/A A/A/B A/B/B B/A/A A/B/B/B B/A/B/B B/B/B/A B/B/A/B A/A/B/B A/B/A/B A/B/B/A B/B/A/A B/A/B/A B/A/A/B A/A/A/B B/A/A/A A/B/A/A A/A/B/A

It is expected that the treatment cycles would be repeated/continued as necessary. It also is contemplated that various standard therapies, as well as surgical intervention, may be applied in combination with the described COX2 inhibitor therapy.

a. ACE Inhibitors

ACE inhibitors are a first class of anti-hypertensives that may be used in combination with COX2 inhibitors as part of the present invention. Such inhibitors include captopril (Capoten™; Bristol-Myers Squibb), benazepril (Lotensin™; Novartis), enalapril (Vasotec™; Merck), fosinopril (Monopril™; Bristol-Myers Squibb), lisinopril (Prinivil™; Merck/Zestril™; Astra-Zeneca), quinapril (Accupril™; Parke-Davis) ramipril (Altace™; Hoechst Marion Roussel, King Pharmaceuticals), imidapril (not approved for human use in the USA; approved in Japan), perindopril erbumine (Aceon™; Rhone-Polenc Rorer) and trandolapril (Mavik™; Knoll Pharmaceutical).

b. ARB

There are seven currently available angiotensin receptor blockers, each of which are suitable for use in combination with COX2 inhibitors as part of the present invention. They are cadesartan (Atacand™; Atacand HCT™), eprosartan (Teveten™), irbesartan (Availide™; Avapro™), losartan (Cozaar™; Hyzaar™), tasosartan (Verdia™), telmisartan (Micardis™) and valsartan (Diovan™; Diovan HCT™).

C. Aldosterone Receptor Antagonist

Another class of drugs suitable for use in combination with COX2 inhibitors in the treatment of ADPKD is aldosterone receptor antagonists. One such compounds is spironolactone, is a synthetic steroid with an aldosterone-like structure, which acts as a competitive antagonist at aldosterone receptors. The most important of these receptors are situated in the distal portion of the renal tubules. Spironolactone thus inhibits sodium and water reabsorption while sparing the potassium and magnesium metabolism. The optimal effect is dependent on a sufficient sodium supply in the distal portion of the renal tubules, as it can be observed in thiazide treatments. Immediate inhibitory effect on aldosterone synthesis is of secondary importance. Spironolactone is also an anti-androgen and is highly efficient against cirrhotic ascites. However, this is almost the only indication where it can be given to men. For all other cases, other drugs should be given preference because of the anti-androgenic effect of spironolactone. In women, the potassium-sparing potential can be exploited to a greater extent.

III. Pharmaceutical Formulations and Routes of Administration

Pharmaceutical compositions of the present invention comprise an effective amount of a COX2 inhibitor dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition containing a COX2 inhibitor will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The pharmaceutical preparation may comprise different types of carriers depending on whether it is to be administered in solid, liquid or aerosol form, and whether it need to be sterile for such routes of administration as injection. The present invention can be administered intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intramuscularly, intraperitoneally, subcutaneously, subconjunctival, intravesicularlly, mucosally, intrapericardially, intraumbilically, intraocularally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The pharmaceutical compositions may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In particular embodiments, the pharmaceutical formulations are prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. Preferred carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain preferred embodiments an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

Additional formulations which are suitable for other modes of administration include suppositories. Suppositories are solid dosage forms of various weights and shapes, usually medicated, for insertion into the rectum, vagina or urethra. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein. In particular embodiments, prolonged absorption of an injectable composition can be brought about by the use in the compositions of agents delaying absorption, such as, for example, aluminum monostearate, gelatin or combinations thereof.

IV. Screening/Diagnosing ADPKD

There are a variety of ways to screen or diagnose ADPKD. First, given the genetic nature of the disease, a careful review of family history can be undertaken. This information typically is obtained through family medical records and from the subject through a patient questionnaire that requests specific information on the health history of his or her relatives.

Renal imaging study has become a common diagnostic tool in ADPKD. The specific kinds of imaging that can be employed to determine the development of cysts include ultrasound, CT scan, MRI, as well as other imaging techniques.

Finally, genetic screening may be employed. As discussed, recent work suggests cyst formation is initiated as a result of a random somatic mutation of the remaining normal PKD allele in patients with germline disruption of one PKD allele (i.e., PKD1+/−) (Koptides et al., 1999; Qian et al., 1996). Analysis of the PKD alleles in cystic cells from ADPKD patients has revealed a loss of heterozygosity (LOH) or intragenic mutations involving the non-affected PKD1 allele in approximately 20% of renal cysts (Qian et al., 1996). Useful techniques for probing changes in chromosomal DNA and mRNA transcripts include RFLP analysis, RT-PCR coupled with sequence analysis, and SNP identification.

### V. EXAMPLES

The following examples are included to demonstrate particular embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Proposed Clinical Trial Outline

The inventor proposes a main intervention trial as per requested by the RFA examining inhibition of the renin angiotensin system, as well as a feasibility trial examining COX2 inhibitors, with an alternate plan to fold the feasibility trial into the main trial as a third arm. This could be advantageous if no established surrogate markers of renal outcome are available. It also would take better advantage of the placebo arm study. This study is a long-term, parallel-group, multicenter, randomized, double-blind comparison of the efficacy and safety of COX2 inhibitors or ACEI in patients with renal insufficiency and ADPKD. Enrollment of 1560 patients is expected to occur in approximately 4 centers over 24 months if a three-arm study is done or 1040 patients if a two arm study is done with ACEI and placebo alone. Patients will be randomized to receive either COX2 inhibitor, ACEI, or placebo with other anti-hypertensive agents excluding ACE1 or angiotensin receptor blockers (ARB) used for uniform BP control (goal <135/85 mmHg). This study consists of 4 periods (A-D) as outlined in the schematic.

Period A is a 2-30 day screening period. During this time, patient eligibility will be determined and a measurement of renal function (serum creatinine and 24-hour creatinine clearance), protein excretion (24-hour urine collection for protein), and blood pressure will be obtained. It is during period A that the patient must meet eligibility criteria for GFR as estimated by the MDRD equation, 24-hour urinary protein excretion (<1000 mg/24 hr) and documentation of ADPKD. Patients who do not initially qualify for randomization may be rescreened in >2 months throughout the enrollment period. The MDRD equation estimating GFR is highly accurate (comparable to iothalamate GFR), and requires only the assessment of serum measurements, height, weight, gender, race, and age. It was derived from a large database of patients one quarter of who had ADPKD. This was chosen patient screening to insure that patients are selected whose GFR falls in the range of 25-55 ml/min/1.73 m2. (MDRD equation for GFR: 170×(Scr)0.999×(age)−0.176×(0.762 for women)×(1.180 for blacks)×(SUN)-0.170×(alb)+0.318 (Levey et al., 1999).

Period B is a 2-30 day washout baseline phase, during which any patients who are already on ACEI, ARBs or COX2 inhibitors will have them discontinued. Patients who were not previously on ACEI or angiotensin II receptor antagonists or COX2 inhibitors will proceed directly to B2 visit after the final screening visit. Interim visits will be scheduled as needed to control blood pressure and insure patient safety. The first of 2 sets of baseline values for renal function will be obtained at the B2 visit (serum creatinine).

Period C occurs 1 day to 3 weeks after the final baseline visit and is the randomization visit where the final baseline assessments of serum creatinine, 24-hour urinary protein excretion and other characteristics are obtained. At this visit, all criteria for randomization are reviewed, and if those criteria are met, the patient will receive the first dose of double-blind study medication, after receiving a data coordinating center randomization assignment. Baseline values for serum creatinine, 24-hour protein excretion, and creatinine clearance will be the mean of those obtained at baseline visit 2 and the randomization visit.

Period D is the double-blind treatment period, during which patients continue to receive study drug. Double-blind study medication will be administered once daily. During the double-blind treatment period, anti-hypertensive therapy will be adjusted to maintain goal blood pressure of 135/85 mmHg in all patients. Other anti-hypertensive agents may be subsequently added as needed to control blood pressure excluding other ACEI or ARB. Patients will be monitored closely for any signs or laboratory evidence of complications related to the use of the study medication during frequent visits immediately following the randomization visit. Once stable, patients will be seen at 35 month intervals for clinical evaluation including recording of adverse experiences, blood pressure measurement, renal function determination, safety laboratory tests, and the assessment of compliance with double-blind medication. Period D will continue until all patients are randomized and the last surviving patient has been followed for a minimum of 24 months. All patients, even those who have reached an endpoint of ESRD or doubling of serum creatinine, will be followed for all visits and will remain on study medication. Those patients who have had study medication stopped will also continue to be followed for all visits.

### Example 2

### Study Population

Source and Number of Patients. A total of 1560 patients meeting eligibility requirements will be randomized in a 1:1:1 ratio, that is, 520 patients will be randomized to COX2 inhibitor and 520 patients will be randomized to ACEI and 520 patients to placebo. (If the COX2 inhibitor arm is chosen by the protocol committee to be reserved 20 for use in a feasibility pilot train then there will be only a 1:1 randomization to ACE1 or placebo in the long-term trial and all other procedures followed as noted below. Each center would enroll 260 patients. It is anticipated that the study will take approximately 4-5 years to conduct: 2 years to complete enrollment followed by a minimum of 2 years of study participation by the last randomized patient. All patients will be seen at all visits regardless of whether or not they are receiving double-blind study medication. Approximately 4 centers will participate. Each center is expected to contribute at least 390 randomized patients to the study. It is expected that all patients will be randomized within 24 months of study initiation.

Patient Selection Criteria. Patients are selected on the following criteria:

>18 y/o

Men or women with documented ADPKD and chronic renal insufficiency, defined as a GFR of 25-55 ml/min/1.73 m2 as estimated by the MDRD equation. (Women must be non-lactating, non-pregnant with adequate contraception.)

Hypertension defined as systolic blood pressure >135 mmHg or diastolic blood pressure >85 mmHg or the use of anti-hypertensive medications.

Willingness and ability to give informed consent and to cooperate with the protocol.

Exclusion Criteria. Patients with the following characteristics are to be excluded:

Serum potassium >5.5 meq/dL.

24 hour urine protein excretion >1000 mg/24 hr.

Evidence that the patient's renal disease has been non-progressive over the 12 months prior to screening (i.e., serum creatinine did not increase or GFR did not decrease by 10% or more during the previous year), if data are available.

The presence of any renal disease other than ADPKD.

Immunosuppressive agents for >2 weeks in the 3 months prior to randomization.

Renovascular disease (uncorrected and hemodynamically significant).

Obstructive uropathy.

History of or evidence of acute renal failure with 6 months prior to randomization visit 1.

Known human immunodeficiency virus disease (HIV).

Other significant abnormalities that the investigator feels may compromise the patient's safety or successful participation in the study.

Anticipated inability to cooperate with or any condition of sufficient severity to impair cooperation in the study.

History of malignancy excluding basal or squamous cell skin cancer or cervical cancer in situ.

Any of the following cardiovascular conditions within one month of the screening visit: a myocardial infarction, coronary angioplasty, coronary artery bypass graft, other revascularization procedure, severe or unstable angina or hemodynamically important vascular heart disease.

Need for chronic (>2 weeks) immunosuppressive therapy, including oral or IV corticosteroids. Inhaled steroids are permissible.

Absolute need for nonsteroidal anti-inflammatory agents or COX2 inhibitors (with the exception of aspirin 81 mg/day).

Absolute need for ACEI or ARB.

History of drug sensitivity or adverse reaction to COX2 inhibitor, ACEI or ARB.

Any medical condition which in the opinion of the investigator will shorten the life of the study participant not previously followed up.

Inability to tolerate oral medication or history of significant malabsorption.

Evidence or suspicion of drug abuse or excessive alcohol consumption within 12 months prior to screening visit 1.

Receipt of any investigational drug within 30 days or 5 half-lives of the investigation drug (the longer period will apply) within screening visit 1.

Absolute need for any prohibited medication specified below.

Inclusion and Exclusion Criteria Justification. Important to note that the average age at which ESRD is reached for ADPKD patients is in the mid 50's. Thus, the population of patients primarily affected by renal failure are elderly. There is no upper age limit on inclusion in this trial since an otherwise healthy, elderly patient with ADPKD is a representative participant in this trial. Patients with CRI are selected so hard clinical outcomes can be observed with sufficient frequency to assess efficacy unless a surrogate such as cyst growth is validated. These accepted hard clinical outcomes are the only currently accepted outcomes. As noted under detailed screening section the MDRD GFR equation is utilized for screening due to the simplicity (no urine measurement, no radioactive substances) using only demographic information, and serum measurements, its accuracy and its development using a large number of ADPKD patients. Patients with >1 gram of protein excretion in the urine are excluded due to the strong possibility that they may have another renal disease in addition to ADPKD. ADPKD documentation requires review by the PI of radiographic imaging reports and the central collection of these reports. Excluded patients include those who are unlikely to complete the study or follow the protocol due to other medical conditions, noncompliance or inability to tolerate or absolute necessity for one of the study drugs.

### Example 3

### Endpoints

Primary. Time from randomization to the first occurrence of doubling of baseline serum creatinine, end stage renal disease, defined as renal transplantation or need for dialysis, or death.

Secondary. Renal:

Occurrence (time-to-event) of each of individual components of the primary composite including death, ESRD or double of serum creatinine.

Occurrence (time-to-event) of the combined endpoint of either end stage renal failure or death.

Change in serum creatinine and/or creatinine clearance.

If available based on results of consortium of radiographic imaging studies of ADPKD a secondary outcome could be based on cyst size or complexity or renal signs.

Tertiary. Safety of the administration of COX2 inhibitors or ACEI on ADPKD-examining serum potassium, acute renal failure and other more standard safety measures.

Dr. Julie Lewis has available to her the detailed data on rate of decline of renal function in ADPKD patients in the MDRD. As noted in detail in the statistical section of the grant the 200 ADPKD patients followed for 2.2 years who had entry GFR's between 25-55 ml/min/1.73 m2 had both a “rapid” rate of decline of renal function and −6 ml/min/yr and had the least variability between patients in rate of decline of renal function compared to other disease categories. This makes it feasible to examine a primary outcome that is a hard clinical outcome of halving GFR (doubling serum creatinine) or ESRD. Deaths are included in the composite outcome to avoid informative censoring.

### Example 4

### Study Visits

Period A (Screening). The purpose of screening is to identify eligible subjects and to exclude ineligible subjects. Initial screening will serve to eliminate unsuitable subjects before the baseline process is started. Thus, a careful history and physical examination will be conducted to ensure that the subjects meets all the inclusion criteria and does not meet any of the exclusion criteria. In addition, the screening period which is from 2 days to 30 days in duration, the following procedures will be performed at the initial screening visit (visit A1):

Informed written consent

Review the inclusion/exclusion criteria

Review/request documentation of ADPKD

Assign subject number

Measure blood pressure, heart rate, height and weight

Blood sampling for serum chemistries (creatinine, albumin, electrolytes, blood urea, nitrogen, potassium, and CBC)

Instruct subjects in the collection of a 24-hour urine

Only subjects who meet all the inclusion and none of the exclusion criteria and whose laboratory analysis does not reveal the presence of any exclusion criteria will proceed to the second screening visit. The procedures to be performed at the second screening visit (day A2):

Review documentation of ADPKD if not done in visit Al

Measure blood pressure, heart rate, and weight

Complete history and physical examination

Review the inclusion and exclusion criteria

Submit 24-hour urine collection for the measurement of urinary protein excretion and creatinine clearance. This urine will serve as the screening urine.

For all visits, a case report form will be completed.

Period B (Baseline). All the results of the laboratory tests shall be reviewed prior to the baseline period visit. Only subjects who fulfill all inclusion/exclusion criteria with documented ADPKD and a GFR between 25-55 ml/min/1.73 m2 as determined by the MDRD equation, and serum potassium <5.5 meq/dL and a 24 hour urine protein excretion <1 gram in 24 hours will continue in the study. All other subjects are to be withdrawn from the study but can be rescreened in 2 months. The baseline period is 2-30 days. 24 hours prior to the B1 visit patients should be instructed to discontinue ACEI, ARB or COX2 inhibitors. Patients not on these medications may proceed directly to the B2 visit and skip the B1 visit.

Day B1 Visit. The procedures and information to be obtained on the first B1 visit include:

Blood pressure, heart rate, and weight

Brief medical history and examination

Adverse event assessment

Instruct patient to collect 24 hour urine for next visit Interim Visits. Between the B1 and B2 visits sufficient interim visits should be scheduled as needed as determined by the investigator for blood pressure control and subject safety. Blood pressure control should be achieved using other anti-hypertensives excluding ACEI or ARBs (see Blood Pressure Management Section). In each interim visits the following procedures will be conducted:

Blood pressure, heart rate, and weight

Brief medical history and evaluation

Adverse event assessment

Visit B2. The B2 visit will between 14-30 days after the B1 visit (if the BI visit was required). The purpose of visit B2 is to insure that the subject meets all the inclusion and none of the exclusion criteria prior to randomization. In addition, it will be assured that the subject's blood pressure is at a safe level to proceed with randomization and the first of 2 baseline laboratory and urinary collections to be made. The procedures that will be obtained include:

Blood pressure, heart rate, and weight

Brief medical history and examination

Blood sampling for serum chemistries and CBC

24-hour urine for protein excretion and creatinine clearance

Adverse event assessment

Period C (Randomization). Only those subjects who fulfill all inclusion and none of the exclusion criteria will proceed to randomization. Also, in order to proceed to the randomization phase of the study, the subject must have a systolic blood pressure at least<160 mmHg and a diastolic blood pressure at least<100 mmHg with a goal of<135/85 mmHg. If the subject has not achieved these blood pressure goals at the end of the baseline period, a third optional month of baseline may be added (prior to B2 visit with laboratory assessments) with sufficient interim visits to achieve this blood pressure goal. The randomization period should occur between 1 day and 3 weeks after the B2 visit. Procedures to occur at randomization include:

Blood pressure, heart rate and weight

Brief medical history and examination

Blood sampling for serum chemistries and CBS

Sample sent to central laboratory for future genetic analysis

24-hour urine protein excretion and creatinine clearance

Adverse event assessment

Assign randomization number

Dispense randomized study drug

Medication dispensing

Laboratory values obtained at the B2 visit and the C1 visit will be averaged and the mean of these values will be used as the baseline values for the subject for the study. At the randomization visit, the subjects will be instructed on medication administration. Subjects will be instructed to take their daily medication once per day throughout the study. Each time medication is dispensed. Compliance will be encouraged. The coordinator will record medications dispensed and returned and do pill counts.

Subject Withdrawal from Double-Blind Medication. Reasons for stopping study medication include:

Subject preference

Hyperkalemia event refractory to corrective measure

Early acute renal failure event refractory to corrective measures

Pregnancy

ESRD

Any clinical adverse event, laboratory abnormality, intercurrent illness or other clinical condition which indicates to the PI that continued participation is not in the best interest of the subject

For subject visits when subjects are being withdrawn from double-blind medication either due to subject preference or due to adverse effects from the medications, all assessments performed at the final visit will be performed. Subjects who have been withdrawn from the study medication, however, will be reminded to return for their next regularly scheduled visit and will continue to return to the clinic on a regular basis until death or the study is completed. If pregnancy or an intercurrent illness resolves, study drug may be resumed. These patients will maintain their original group assignment. All study drug discontinuations and resumptions must be approved by the Clinical Management Committee.

### Example 5

### Patient Restrictions and Characteristics

Prohibited Medications or Precautions. The following drug restrictions and precautions are provided:

Nonsteroidal anti-inflammatory agents or COX2 inhibitors with the exception of aspirin (up to 81 mg/day).

Other ACEI or ARB

Agents that inhibit the tubular secretion of creatinine (cimetidine, trimethoprin) should not be used when creatinines are obtained

Systemic steroids for>2 weeks

Any systemic immunosuppressive agent (e.g., cyclosporine)

Should NSAIDS, COX2 inhibitors, ACEI or ARB be necessary during the course of the trial, study drug should be temporarily withheld and restarted only after time has elapsed to allow for sufficient clearance of medication. In addition, temporarily withholding study drug should be considered in any patient with a risk factor predisposing to the development of renal failure.

Allowable concurrent medications. The following drugs are permitted:

The dosage and regimen of any chronic, permitted concurrent medications (e.g._blockers, thiazides, loop diuretics, H2 antagonists, aspirin, phenytoin) should be stable at least 30 days prior to the screening visit.

Any medications prescribed chronically or intermittently during the course of the study, should have the dose and dose adjustments of these medications reported on the concurrent medication CRF.

Blood Pressure Control/Management of Hypertension and Edema. This study is designed to attain equal degrees of blood pressure control within both treatment groups by the use of target blood pressure goals. Office seated blood pressure readings will be used for making therapeutic decisions. The investigator is free to use his/her best clinical judgment regarding adjustments of anti-hypertensive dosage to achieve the blood pressure goals. The systolic blood pressure goal is 135 mmHg and the diastolic blood pressure goal is 85 mmHg. Other anti-hypertensive agents excluding other ACEI or ARB should be used as needed to control blood pressure. It is anticipated that the investigators will use good clinical judgment in choosing anti-hypertensive medications to achieve the blood pressure goals. Since many of these patients have renal insufficiency, diuretics may be very important in managing their salt and water excess and maintaining adequate blood pressure control. During all the study period, the use of adjunctive anti-hypertensive medications is permitted to maintain blood pressure control. Additional visits to optimize blood pressure control may be scheduled as needed during the screening period. It is critical that these patients have adequate blood pressure control and that sufficient interim visits are completed to achieve the blood pressure goals at each and every 3-month visit, if possible. If at any time during the course of the study the blood pressure (systolic>180 or diastolic>110) control is refractory to treatment or the edema is refractory to treatment after consultation with the Clinical Management Committee, the study drug may be stopped.

Doubling of Serum Creatinine. Doubling of serum creatinine is one of the key elements of the primary composite outcome. The baseline creatinine is defined as the mean of the two serum creatinine levels obtained at the second baseline and randomization visit. These creatinines will be measured in a central laboratory. If the patient is found to have a serum creatinine measured in the central laboratory, twice the baseline value the patient should be evaluated within one week for medications (Bactrim, Cimetidine) or events (obstruction, urinary tract infection) which may alter serum creatinine and corrective measures taken. After corrective measures (if indicated) within 1-4 weeks a second serum creatinine must be sent to the central laboratory for confirmation. If this repeat serum creatinine is still two times of serum creatinine event is then confirmed. Study drug will be continued after doubling of serum creatinine until the patient reaches ESRD. Study visits will also continue.

Other Renal Outcomes. Other renal outcomes commonly seen in patients with ADPKD will also be documented. A patient will be classified as having a kidney stone if they have documentation of passing a kidney stone or radiographic evidence of a new stone. A urinary tract infection requires documentation by urine culture. Gross hematuria requires confirmation by urine analysis and cyst rupture requires radiographic evidence. Flank pain will require precipitation of a hospital admission for this.

Hyperkalemia. If at any time the serum potassium increases 0.5 meq/L and is >5.0 meq/L confirmed by repeat measurement corrective measures must be initiated to prevent further increases in serum potassium, including review of the patients dietary intake and review of medications that may be contributing to hyperkalemia. Also diuretics and/or sodium polystyrene sulfonate may be administered as needed. If during any follow up visit, the patient's serum potassium is >6.0 meq/L, the investigator will schedule an interim visit within 48 hours and take immediate measures to determine the cause (e.g., patient use K+ containing salt substitute) and take corrective action which may include stopping the study drug. The patient should be scheduled for a return visit in 5 days or less and the potassium rechecked. If it is less than 5.5 meq/L and study drug has not been discontinued the patient can continue on study drug and resume normal visits. If the potassium does not correct to 5.5 meq/L or less and study drug has not been discontinued it should be and an interim visit scheduled. If the serum potassium corrects to 5.5 meq/L or less and study drug has been discontinued the patient can restart study drug, if the principal investigator in conjunction with the clinical management committee determine it is clinically indicated to do so. The serum potassium should be checked weekly for three weeks. If serum potassium rises then study drug will be discontinued. Persistent hyperkalemia (serum potassium >6.0 meq/L) refractory to corrective measures requires permanent discontinuation of the study drug.

Early Creatinine Rise. This phenomenon is being defined because of the possibility that rare patients entered into the study may have an adverse glomerular hemodynamic event because of the use of the ACEI or COX2 inhibitor. Patients entered into the randomized study will have their serum creatinine checked by the clinical laboratory at the 4 day and 2, 4, 8 and 12 week schedule follow up visits. If the serum creatinine rises greater than 50% of the baseline serum creatinine with an increase of at least 1.0 mg/dL, the patient is defined as having an early creatinine rise event. The investigator should begin evaluating the Early Creatinine Review event and report it. If the patient is taking medications which alter serum creatinine, or if any of the following conditions are present:

plasma volume depletion

infected urine

obstructive uropathy, or

decreased cardiac output

The investigator should take corrective measures and schedule an interim visit one week from the institution of the measures. If the serum creatinine measured during this interim visit again is 50% greater than the baseline serum creatinine with an increase of at least 1.0 mg/dL, the study medication is stopped. If the patient is not taking medications which alter serum creatinine and does not have any of the conditions listed above, the investigator should schedule an interim visit as soon as possible. If the serum creatinine measured during this interim visit is again 50% greater than the baseline serum creatinine recorded with an increase of at least 1.0 mg/dL, the study medication is stopped and the patient must be considered for diagnostic work up.

Documentation. The identification of potential clinical outcomes will be made by the study site and communicated to the data coordinating center immediately (within 24 hours). Obtaining the documentation required to support endpoint adjudication by the Outcome Committee will be the responsibility of the study site. Endpoint documentation will include, but is not limited to results of laboratory tests, hospital discharge summaries, clinic notes, results of other diagnostic tests, autopsy reports and death certificate information.

### Example 6

### Safety

Evaluation of the safety of COX2 inhibitors and ACEI will be based upon the assessment of adverse events and clinically important changes in laboratory parameters (serum potassium, serum creatinine). Particular attention will be given to those events which result in discontinuation of study drug or which are serious in nature.

Laboratory Tests. All laboratory tests will be done in the central laboratory except for those done urgently at the PCC for safety reasons:

Serum chemistries will include: blood urea nitrogen, creatinine, albumin, electrolytes, SGOT, cholesterol, alkaline phosphatase glucose, total bilirubin

CBC will include a white blood cell count, RBC indices, hemoglobin, and hematocrit

24 hour urine tests will include: total volume, creatinine, protein

Aliquots will be stored for genetic analysis in the future.

Both the 24 hour urine studies and blood for genetic analysis may be stored and saved for testing as an ancillary study funded through a separate R01 mechanism if monies in this RFA are short. This will allow the development of a specimen bank.

Adverse Event Reporting. Each patient will be observed and queried in a nonspecific fashion at each visit after screening visit 1 during the study for any new or continuing symptoms since the previous visit. During the double-blind period of this trial, the investigational study drugs are ACEI and COX2 inhibitors. Any adverse event is any reaction, side effect or other undesirable event that occurs in conjunction with the use of a drug, biological product or diagnostic agent in humans whether or not the event is considered drug related. In this study, this includes any illness, sign, symptom or clinically significant laboratory test abnormality that has appeared or worsened during the course of the clinical trial, regardless of cause or relationship to the drugs under study. Patients experiencing adverse events that cause interruption or discontinuation of the study drug or those experiencing adverse events that are present at the end of their participation in the study should receive follow up as appropriate. If possible, a report of the outcome of any adverse event present at the end of the study that was considered by the investigator to be related, probably or possibly related or of undetermined relationship to study drug should be made.

Serious Adverse Events. An event that is serious must be recorded on the serious adverse event (SAE) and required expeditious handling. Potentially SAEs should be followed to resolution or stabilization and reported as SAEs if they become serious. A SAE is one that meets one of the following criteria:

fatal

life threatening

permanently disabling

requiring or prolonging in-patient hospitalization

congenital anomaly, cancer, or overdose

medically significant event (includes laboratory abnormalities)

Planned Safety Evaluation. All patients who received at least one study dose of medication will be assessed for clinical safety and tolerability. Evaluation of safety data will be based on comparison of patient experience by medication regiment. Clinical interpretation of safety will be based on reviews of adverse experiences, vital signs, and laboratory test values. The instance of adverse experiences according to time to onset and summary statistics of vital signs, and laboratory test values will be presented to a Data Safety Monitoring Board (DSMB). This data will be evaluated by the DSMB at planned interim analyses. The DSMB will meet at 6 months, 12 months and every year thereafter to review the accumulated study data concerning patient safety. Interim statistical reports will be prepared under the direction of the biostatistical coordinating center for review by the DSMB. The timing of these meetings are subject to change based on the results of the interim reports. Each interim report will be unblinded and will include tabulations and analyses of patient baseline characteristics, patient withdrawals and all major clinical adverse experience by medication regiment including those that may have occurred after a patient has reached an endpoint and discontinued study medication. In reviewing each interim report, the DSMB will assess the need to perform a detailed evaluation of the benefits and risks of continuing the study based on the data on the interim report.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

Agodoa, L. Y., Appel, L., Bakris, G. L., Beck, G., Bourgoignie, J., Briggs, J. P., Charleston, J., Cheek, D., Cleveland, W., Douglas, J. G., Douglas, M., Dowie, D., Faulkner, M., Gabriel, A., Gassman, J., Greene, T., Hall, Y., Hebert, L., Hiremath, L., Jamerson, K., Johnson, C. J., Kopple, J., Kusek, J., Lash, J., Lea, J., Lewis, J. B., Lipkowitz, M., Massry, S., Middleton, J., Miller, E. R., 3rd, Norris, K., O'Connor, D., Ojo, A., Phillips, R. A., Pogue, V., Rahman, M., Randall, O. S., Rostand, S., Schulman, G., Smith, W., Thomley-Brown, D., Tisher, C. C., Toto, R. D., Wright, J. T., Jr., and Xu, S. (2001). Effect of ramipril vs amlodipine on renal outcomes in hypertensive nephrosclerosis: a randomized controlled trial. Jama 285, 2719-28.

Arias, A. M. (2001). Epithelial mesenchymal interactions in cancer and development. Cell 105, 425-31.

Bajwa, Z. H., Gupta, S., Warfield, C. A., and Steinman, T. I. (2001). Pain management in polycystic kidney disease. Kidney Int. 60, 1631-1644.

Bamba, H., Ota, S., Kato, A., Adachi, A., Itoyama, S., and Matsuzaki, F. (1999). High expression of cyclooxygenase-2 in macrophages of human colonic adenoma. Int J Cancer 83, 470-5.

Bello-Reuss, E., Holubec, K., and Rajaraman, S. (2001). Angiogenesis in autosomal-dominant polycystic kidney disease. Kidney Int 60, 37-45.

Bernstein, J., Evan, A. P., and Gardner, K. D., Jr. (1987). Epithelial hyperplasia in human polycystic kidney diseases. Its role in pathogenesis and risk of neoplasia. Am J Pathol 129, 92-101.

Brater, D. C. (1999). Effects of nonsteroidal anti-inflammatory drugs on renal function: focus on cyclooxygenase-2-selective inhibition. Am J Med 107, 65S-70S; discussion 70S-71 S.

Brenner, B. M., Cooper, M. E., de Zeeuw, D., Keane, W. F., Mitch, W. E., Parving, H.-H., Remuzzi, G., Snapinn, S. M., Zhang, Z., Shahinfar, S., and the RENAAL Study Investigators (2001). Effects of Losartan on Renal and Cardiovascular Outcomes in Patients with Type 2 Diabetes and Nephropathy. N Engl J Med 345,861-869.

Breyer, M. D., and Harris, R. C. (2001). Cyclooxygenase 2 and the kidney. Curr Opin Nephrol Hypertens 10, 89-98.

Chapman, A. B., Johnson, A., Gabow, P. A., and Schrier, R. W. (1990). The renin-angiotensin-aldosterone system and autosomal dominant polycystic kidney disease. N Engl J Med 323, 1091-6.

Chapman, A. B., Johnson, A. M., Gabow, P. A., and Schrier, R. W. (1994). Overt proteinuria and microalbuminuria in autosomal dominant polycystic kidney disease. J Am Soc Nephrol 5, 1349-54.

Chapple, K. S., Cartwright, E. J., Hawcroft, G., Tisbury, A., Bonifer, C., Scott, N., Windsor, A. C., Guillou, P. J., Markham, A. F., Coletta, P. L., and Hull, M. A. (2000). Localization of cyclooxygenase-2 in human sporadic colorectal adenomas. Am J Pathol 156, 545-53.

Contreras, G., Mercado, A., Pardo, V., and Vaamonde, C. A. (1995). Nephrotic syndrome in autosomal dominant polycystic kidney disease. J Am Soc Nephrol 6, 1354-9.

Davis, I. D., MacRae Dell, K., Sweeney, W. E., and Avner, E. D. (2001). Can progression of autosomal dominant or autosomal recessive polycystic kidney disease be prevented? Semin Nephrol 21, 430-40.

Ecder, T., Edelstein, C. L., Fick-Brosnahan, G. M., Johnson, A. M., Chapman, A. B., Gabow, P. A., and Schrier, R. W. (2001). Diuretics versus angiotensin-converting enzyme inhibitors in autosomal dominant polycystic kidney disease. Am J Nephrol 21, 98-103.

Ecder, T., and Schrier, R. W. (2001). Hypertension in autosomal-dominant polycystic kidney disease: early occurrence and unique aspects. J Am Soc Nephrol 12, 194-200.

Fick, G. M., and Gabow, P. A. (1994). Natural history of autosomal dominant polycystic kidney disease. Annu Rev Med 45, 23-9.

Gabow, P. A. (1993). Autosomal dominant polycystic kidney disease. N Engl J Med 329, 332-42.

Gabow, P. A., Johnson, A. M., Kaehny, W. D., Kimberling, W. J., Lezotte, D. C., Duley, I. T., and Jones, R. H. (1992). Factors affecting the progression of renal disease in autosomal-dominant polycystic kidney disease. Kidney Int 41, 1311-9.

Geberth, S., Stier, E., Zeier, M., Mayer, G., Rambausek, M., and Ritz, E. (1995). More adverse renal prognosis of autosomal dominant polycystic kidney disease in families with primary hypertension. J Am Soc Nephrol 6, 1643-8.

Giardiello, F. M., Hamilton, S. R., Krush, A. J., Piantadosi, S., Hylind, L. M., Celano, P., Booker, S. V., Robinson, C. R., and Offerhaus, G. J. (1993). Treatment of colonic and rectal adenomas with sulindac in familial adenomatous polyposis. N Engl J Med 328, 1313-6.

Giovannucci, E., Egan, K., Hunter, D., Stampfer, M., Colditz, G., Willett, W., and Speizer, F. (1995). Aspirin and the risk of colorectal cancer in women. N Engl J Med 333, 609-14.

Graham, P. C., and Lindop, G. B. (1988). The anatomy of the renin-secreting cell in adult polycystic kidney disease. Kidney Int 33, 1084-90.

Grantham, J. J. (1997). Mechanisms of progression in autosomal dominant polycystic kidney disease. Kidney Int Suppl 63, S93-7.

Hao, C. M., Yull, F., Blackwell, T., Komhoff, M., Davis, L. S., and Breyer, M. D. (2000). Dehydration activatesan NF-kappaB-driven, COX2-dependent survival mechanism in renal medullary interstitial cells. J Clin Invest 106, 973-82.

Harding, P., Carretero, O. A., and Beierwaltes, W. H. (2000). Chronic cyclooxygenase-2 inhibition blunts low sodium-stimulated renin without changing renal haemodynamics. J Hypertens 18, 1107-13.

Hardwick, J. C., van den Brink, G. R., Offerhaus, G. J., van Deventer, S. J., and Peppelenbosch, M. P. (2001). NF-kappaB, p38 MAPK and JNK are highly expressed and active in the stroma of human colonic adenomatous polyps. Oncogene 20, 819-27.

Harris, R. C., and Breyer, M. D. (2001). Physiological regulation of cyclooxygenase-2 in the kidney. Am J Physiol Renal Physiol 281, F1-11.

Harris, R. C., McKanna, J. A., Akai, Y., Jacobson, H. R., Dubois, R. N., and Breyer, M. D. (1994). Cyclooxygenase-2 is associated with the macula densa of rat kidney and increases with salt restriction. J Clin Invest 94, 2504-10.

Imanishi, M., Kawamura, M., Akabane, S., Matsushima, Y., Kuramochi, M., Ito, K., Ohta, M., Kimura, K., Takamiya, M., and Omae, T. (1989). Aspirin lowers blood pressure in patients with renovascular hypertension. Hypertension 14, 461-468.

Jackson, E. (1989). Relationship between renin release and blood pressure response to nonsteroidal antiinflammatory drugs in hypertension. Hypertension 14, 469-471.

Jackson-Grusby, L., Beard, C., Possemato, R., Tudor, M., Fambrough, D., Csankovszki, G., Dausman, J., Lee, P., Wilson, C., Lander, E., and Jaenisch, R. (2001). Loss of genomic methylation causes p53-dependent apoptosis and epigenetic deregulation. Nat Genet 27, 31-9.

Keith, D. S., Torres, V. E., Johnson, C. M., and Holley, K. E. (1994). Effect of sodium chloride, enalapril, and losartan on the development of polycystic kidney disease in Han:SPRD rats. Am J Kidney Dis 24, 491-8.

Kennefick, T. M., Al-Nimri, M. A., Oyama, T. T., Thompson, M. M., Kelly, F. J., Chapman, J. G., and Anderson, S. (1999). Hypertension and renal injury in experimental polycystic kidney disease. Kidney Int 56, 2181-90.

Kim, E., Arnould, T., Sellin, L. K., Benzing, T., Fan, M. J., Gruning, W., Sokol, S. Y., Drummond, I., and Walz, G. (1999). The polycystic kidney disease 1 gene product modulates Wnt signaling. J Biol Chem 274, 4947-53.

Kinzler, K. W., and Vogelstein, B. (1996). Lessons from hereditary colorectal cancer. Cell 87, 159-70.

Klahr, S., Breyer, J. A., Beck, G. J., Dennis, V. W., Hartman, J. A., Roth, D., Steinman, T. I., Wang, S. R., and Yamamoto, M. E. (1995). Dietary protein restriction, blood pressure control, and the progression of polycystic kidney disease. Modification of Diet in Renal Disease Study Group [published erratum appears in J Am Soc Nephrol 1995 Oct;6(4):1318]. J Am Soc Nephrol 5, 2037-47.

Klahr, S., Levey, A. S., Beck, G. J., Caggiula, A. W., Hunsicker, L., Kusek, J. W., and Striker, G. (1994). The effects of dietary protein restriction and blood-pressure control on the progression of chronic renal disease. Modification of Diet in Renal Disease Study Group [see comments]. N Engl J Med 330, 877-84.

Komhoff, M., Guan, Y., Shappell, H. W., Davis, L., Jack, G., Shyr, Y., Koch, M. O., Shappell, S. B., and Breyer, M. D. (2000). Enhanced expression of cyclooxygenase-2 in high grade human transitional cell bladder carcinomas. Am J Pathol 157, 29-35.

Kömhoff, M., Guan, Y., Shappell, H. W., Davis, L., Jack, G., Shyr, Y., Koch, M. O., Shappell, S. B., and Breyer, M. D. (2000). Enhanced expression of cyclooxygenase-2 in high grade human transitional cell bladder carcinomas. Am J Pathol 157, 29-35.

Komhoff, M., Jeck, N. D., Seyberth, H. W., Grone, H. J., Nusing, R. M., and Breyer, M. D. (2000). Cyclooxygenase-2 expression is associated with the renal macula densa of patients with Bartter-like syndrome. Kidney Int 58, 2420-4.

Koptides, M., Hadjimichael, C., Koupepidou, P., Pierides, A., and Constantinou Deltas, C. (1999). Germinal and somatic mutations in the PKD2 gene of renal cysts in autosomal dominant polycystic kidney disease. Hum Mol Genet 8, 509-13.

Korinek, V., Barker, N., Morin, P. J., van Wichen, D., de Weger, R., Kinzler, K. W., Vogelstein, B., and Clevers, H. (1997). Constitutive transcriptional activation by a beta-catenin-Tcf complex in APC−/− colon carcinoma. Science 275, 1784-7.

Lachin, J. M., and Foulkes, M. A. (1986). Evaluation of sample size and power for analyses of survival with allowance for nonuniform patient entry, losses to follow-up, noncompliances, and stratification. Biometrics 42, 507-519.

Levey, A. S., Bosch, J. P., Lewis, J. B., Greene, T., Rogers, N., and Roth, D. (1999). A more accurate method to estimate glomerular filtration rate from serum creatinine: a new prediction equation. Modification of Diet in Renal Disease Study Group. Ann Intern Med 130, 461-70.

Lewis, E. J., Hunsicker, L. G., Bain, R. P., and Rohde, R. D. (1993). The effect of angiotensin-convertingenzyme inhibition on diabetic nephropathy. The Collaborative Study Group. N Engl J Med 329, 1456-62.

Lewis, E. J., Hunsicker, L. G., Clarke, W. R., Berl, T., Pohl, M. A., Lewis, J. B., Ritz, E., Atkins, R. C., Rohde, R., Raz, I., and the Collaborative Study Group (2001). Renoprotective Effect of the Angiotensin-Receptor Antagonist Irbesartan in Patients with Nephropathy Due to Type 2 Diabetes. N Engl J Med 345, 851-860.

Liotta, L. A., and Kohn, E. C. (2001). The microenvironment of the tumour-host interface. Nature 411, 375-9.

Marcus, A. (1995). Aspirin as prophylaxis against colorectal cancer. N Engl J. Med 333, 656-658.

Maschio, G., Alberti, D., Janin, G., Locatelli, F., Mann, J. F., Motolese, M., Ponticelli, C., Ritz, E., and Zucchelli, P. (1996). Effect of the angiotensin-converting-enzyme inhibitor benazepril on the progression of chronic renal insufficiency. The Angiotensin-Converting-Enzyme Inhibition in Progressive Renal Insufficiency Study Group. N Engl J Med 334, 939-45.

Masferrer, J. L., Leahy, K. M., Koki, A. T., Zweifel, B. S., Settle, S. L., Woemer, B. M., Edwards, D. A., Flickinger, A. G., Moore, R. J., and Seibert, K. (2000). Antiangiogenic and antitumor activities of cyclooxygenase-2 inhibitors [In Process Citation]. Cancer Res 60, 1306-11.

Morin, P. J., Sparks, A. B., Korinek, V., Barker, N., Clevers, H., Vogelstein, B., and Kinzler, K. W. (1997). Activation of beta-catenin-Tcf signaling in colon cancer by mutations in beta-catenin or APC. Science 275, 1787-90.

Nadasdy, T., Laszik, Z., Lajoie, G., Blick, K. E., Wheeler, D. E., and Silva, F. G. (1995).

Proliferative activity of cyst epithelium in human renal cystic diseases. J Am Soc Nephrol 5, 1462-8.

Ogbom, M. R., Sareen, S., and Pinette, G. (1995). Cilazapril delays progression of hypertension and uremia in rat polycystic kidney disease. Am J Kidney Dis 26, 942-6.

Ong, A. C., Ward, C. J., Butler, R. J., Biddolph, S., Bowker, C., Torra, R., Pei, Y., and Harris, P. C. (1999). Coordinate expression of the autosomal dominant polycystic kidney disease proteins, polycystin-2 and polycystin-1, in normal and cystic tissue. Am J Pathol 154, 1721-9.

Oshima, M., Dinchuk, J. E., Kargman, S. L., Oshima, H., Hancock, B., Kwong, E., Trzaskos, J. M., Evans, J. F., and Taketo, M. M. (1996). Suppression of intestinal polyposis in Apc delta716 knockout mice by inhibition of cyclooxygenase 2 (COX-2). Cell 87, 803-9.

Oshima, M., Murai, N., Kargman, S., Arguello, M., Luk, P., Kwong, E., Taketo, M. M., and Evans, J. F. (2001). Chemoprevention of intestinal polyposis in the Apcdelta716 mouse by rofecoxib, a specific cyclooxygenase-2 inhibitor. Cancer Res 61, 1733-40.

Powell, S. M., Petersen, G. M., Krush, A. J., Booker, S., Jen, J., Giardiello, F. M., Hamilton, S. R., Vogelstein, B., and Kinzler, K. W. (1993). Molecular diagnosis of familial adenomatous polyposis. N Engl J Med 329, 1982-7.

Qian, F., Watnick, T. J., Onuchic, L. F., and Germino, G. G. (1996). The molecular basis of focal cyst formation in human autosomal dominant polycystic kidney disease type I. Cell 87, 979-87.

Qian, Q., Harris, P. C., and Torres, V. E. (2001). Treatment prospects for autosomal-dominant polycystic kidney disease. Kidney Int 59, 2005-22. Ramasubbu, K., Gretz, N., and Bachmann, S. (1998). Increased epithelial cell proliferation and abnormal extracellular matrix in rat polycystic kidney disease. J Am Soc Nephrol 9, 937-45.

Ruggenenti, P., Perna, A., Gherardi, G., Benini, R., and Remuzzi, G. (2000). Chronic proteinuric nephropathies: outcomes and response to treatment in a prospective cohort of 352 patients with different patterns of renal injury. Am J Kidney Dis 35, 1155-65.

Ruggenenti, P., Perna, A., Zoccali, C., Gherardi, G., Benini, R., Testa, A., and Remuzzi, G. (2000). Chronic proteinuric nephropathies. II. Outcomes and response to treatment in a prospective cohort of 352 patients: differences between women and men in relation to the ACE gene polymorphism. Gruppo Italiano di Studi Epidemologici in Nefrologia (Gisen). J Am Soc Nephrol 11, 88-96.

Shiff, S. J., and Rigas, B. (1999). Aspirin for cancer [news; comment]. Nat Med 5, 1348-9.

Steinbach, G., Lynch, P. M., Phillips, R. K., Wallace, M. H., Hawk, E., Gordon, G. B., Wakabayashi, N., Saunders, B., Shen, Y., Fujimura, T., Su, L. K., and Levin, B. (2000). The effect of celecoxib, a cyclooxygenase-2 inhibitor, in familial adenomatous polyposis. N Engl J Med 342, 1946-52.

Su, L. K., Vogelstein, B., and Kinzler, K. W. (1993). Association of the APC tumor suppressor protein with catenins. Science 262, 1734-7.

System, U. R. D. (1999). USRDS 1999 Annual Data Report. National Institutes of Health, National Institute of Diabetes digestive and kidney diseases, Bethesda MD.

Torres, V. E., Donovan, K. A., Scicli, G., Holley, K. E., Thibodeau, S. N., Carretero, O. A., Inagami, T., McAteer, J. A., and Johnson, C. M. (1992). Synthesis of renin by tubulocystic epithelium in autosomaldominant polycystic kidney disease. Kidney Int 42, 364-73.

van Adelsberg, J. (2000). Polycystin-1 interacts with E-cadherin and the catenins—clues to the pathogenesis of cyst formation in ADPKD? [editorial]. Nephrol Dial Transplant 15, 1-2.

Wang, J. L., Cheng, H. F., and Harris, R. C. (1999). Cyclooxygenase-2 inhibition decreases renin content and lowers blood pressure in a model of renovascular hypertension. Hypertension 34, 96-101.

Williams, C. S., Mann, M., and DuBois, R. N. (1999). The role of cyclooxygenases in inflammation, cancer, and development. Oncogene 18, 7908-16.

Woo, D. (1995). Apoptosis and loss of renal tissue in polycystic kidney diseases. N. Engl. J. Med. 333, 18-24.

What is claimed is:

1. A method of preventing or treating autosomal dominant polycystic kidney disease (ADPKD) in a subject comprising administering to said subject an effective amount of a cyclooxygenase 2 (COX2) inhibitor.

2. The method of claim 1, wherein said COX2 inhibitor is selected from the group consisting of celecoxib, rofecoxib, paracoxib, valdecoxib, etoricoxib, meloxicam, or nimesulide.

3. The method of claim 1, wherein said method is preventing ADPKD in said subject prior to the development of symptomatic renal disease.

4. The method of claim 3, wherein said method comprises administration of said COX2 inhibitor, beginning at early adulthood, to said subject, said subject having been determined to be at risk of ADPKD as determined by family history, renal imaging study and/or genetic screening.

5. The method of claim 1, wherein said method comprises treating ADPKD in said subject exhibiting symptomatic renal disease and comprises slowing or halting disease progression.

6. The method of claim 5, wherein said subject also suffers from chronic renal insufficiency.

7. The method of claim 1, wherein the dose of said COX2 inhibitor is adjusted to avoid unwanted kidney blood flow effects, thereby maintaining normal renal function and blood pressure.

8. The method of claim 1, further comprising administering to said subject a second drug.

9. The method of claim 8, wherein said second drug is an anti-hypertensive.

10. The method of claim 9, wherein said anti-hypertensive is a ACE inhibitor, an angiotensin receptor blocker, or an aldosterone receptor antagonists.

11. The method of claim 1, wherein said subject is a non-human animal.

12. The method of claim 1, wherein said subject is a human.

13. The method of claim 1, further comprising monitoring one or more conditions in said subject following administration of said COX2 inhibitor.

14. The method of claim 13, wherein said one or more conditions comprise blood pressure, serum creatinine, blood urea nitrogen, urinary protein excretion, or glomerular filtration rate.

15. The method of claim 1, wherein said subject has or will receive therapeutic nephrectomy.

16. The method of claim 15, wherein said therapeutic nephrectomy occurs before COX2 inhibitor administration.

17. The method of claim 15, wherein said therapeutic nephrectomy occurs after COX2 inhibitor administration.
